(19) United States
(12) Patent Application Publication
(10) Pub. No.: US 2023/0092138 A1
(43) Pub. Date: Mar. 23, 2023

(54) EXTERNAL CIRCULATION RENEWAL DEVICE OF CULTURE MEDIA AND VENTILATION DEVICE FOR BIOREACTOR

(71) Applicant: ALIT BIOTECH (SHANGHAI) CO., LTD., Shanghai (CN)

(72) Inventors: Yu LIU, Shanghai (CN); Rui CHEN, Shanghai (CN)

(73) Assignee: ALIT BIOTECH (SHANGHAI) CO., LTD., Shanghai (CN)

(21) Appl. No.: 18/058,859

(22) Filed: Nov. 26, 2022

Related U.S. Application Data
(63) Continuation-in-part of application No. PCT/CN2021/073800, filed on Jan. 26, 2021.
Application No. PCT/CN2021/073800 is a continuation-in-part of application No. PCT/CN2021/073796, filed on Jan. 26, 2021.

(30) Foreign Application Priority Data
May 26, 2020 (CN) 202010457769.1
May 26, 2020 (CN) 202010457772.3

(51) Int. Cl. C12M 1/00; C12M 1/34; C12M 1/26

(52) U.S. Cl. CPC C12M 29/06; C12M 29/04; C12M 41/26; C12M 41/34; C12M 33/14

(57) ABSTRACT

The present disclosure provides a bioreactor, which includes a tank configured to contain a mixture of animal cells and a liquid; and a first ventilation device arranged outside the tank and including a first gas distributor, the first gas distributor being configured to transmit a gas to the liquid separated from the animal cells. The bioreactor further includes a dialysis component, which is arranged outside the tank and comprises a dialysis filter. The bioreactor further includes a second ventilation device arranged in the tank and configured to transmit the gas to the mixture in the tank.

### CROSS REFERENCE TO RELATED APPLICATION

This application is a continuation-in-part of International Application No. PCT/CN2021/073796, filed on Jan. 26, 2021, which claims priority to Chinese Patent Application No. 202010457769.1, filed on May 26, 2020. This application is also a continuation-in-part of International Application No. PCT/CN2021/073800, filed on Jan. 26, 2021, which claims priority to Chinese Patent Application No. 202010457772.3, filed on May 26, 2020. The entire contents of all the above applications are incorporated herein by reference.

### TECHNICAL FIELD

The present disclosure generally relates to the field of biological devices, and more specifically, to bioreactors.

### BACKGROUND

The solubility of oxygen in the culture medium is always one of the most important parameters to design animal cell bioreactors. In general, the oxygen transmission rate is greater than that of animal cells to maintain the normal metabolic level of animal cells. In order to improve the specific surface area of mass transfer under the unit amount of gas and increase the efficiency of oxygen transfer, various methods have been adopted to ensure that the air bubbles made by bioreactors are as small as possible. However, studies found that the smaller the bubbles, the greater the shear force generated by the bubble rupture in the rising process, which can have an irreversible impact on the vulnerable animal cells.

At present, most disposable bioreactors use the combination of centimeter-scale bubble distributors and micron-scale bubble distributors. Compared with the micron-scale bubble distributors, the diameters of bubbles generated by the centimeter-scale bubble distributors are larger and the specific surface areas of mass transfer are smaller. Therefore, the centimeter-scale bubble distributors have a better ability to discharge carbon dioxide, but a poorer ability to transfer oxygen, and they are generally used to disperse excess carbon dioxide bubbles dissolved in the culture medium. The micron-scale bubble distributor is usually made by a sintering method of metal or plastic materials, and its minimum pore diameter may be controlled to 2-20 um. Under the same gas flow conditions, the micron-scale bubble distributor produces a large number of bubbles and effectively improves the specific surface area of mass transfer. However, small bubbles produced by the micron-scale bubble distributor sometimes cause significant damage to animal cells (especially stem cells). Therefore, in the design of the bioreactor, especially for animal cells, the contradiction between oxygen transfer and bubble shear force is more prominent.

The bubble-free design is important for the bioreactor for animal cells. However, the existing bubble-free bioreactors usually adopt a mode of mass transfer in the external circulation of a reactor, use a peristaltic pump to pump the culture medium out of the culture system, and increase the shear damage of the peristaltic pump to animal cells. In addition, the existing bioreactors are mainly stainless-steel tanks, which need damp-heat sterilization in the process of use, so it is difficult to apply many newly developed microporous membrane materials.

In the design of bioreactor for animal cells, how to eliminate harmful substances in culture medium is also a difficult problem. Animal cells metabolize a large number of by-products, and the accumulation of by-products has a great inhibitory effect on the growth and metabolism of animal cells. Therefore, it is necessary to monitor the concentration of such by-products and find ways to discharge such by-products from the culture medium. At present, most disposable bioreactors use perfusion (dilution or gravity sedimentation for liquid exchange) to separate animal cells from the culture medium and inject fresh culture medium. Although this method effectively dilutes the concentration of by-products, it also brings two problems. One is the massive waste of culture medium. Specifically, one to two culture medium volumes are needed to be changed daily, and if 14 days are used as a cycle of the whole process, 10-20 culture medium volumes may be lost. The price of animal cell culture medium (especially stem cell culture medium) is generally one thousand to several thousand yuan per liter, and the cost is huge in the large-scale cultivation. The other is the loss of effective growth factors. Specifically, the culture medium is replaced by using a direct dilution method, which not only dilutes the by-products but also dilutes the concentration of growth factors that promote cell growth. Additional growth factors can be added, but the configuration is complex and the cost is very high.

On the other hand, it is very important to monitor the oxygen consumption rate of animal cells during operation of the bioreactor. The common practice is to pass the tail gas of the bioreactor into the gas mass spectrometer detector. The oxygen uptake rate (OUR) consumed by animal cells is inferred by comparing the inlet gas flow and its gas composition, and the outlet gas flow and its gas composition. However, in the process of gas outlet, the non-liquid area above the bioreactor may form a tail gas buffer zone, making the calculation of OUR invalid and it is difficult to obtain the real-time value of OUR. In addition, the gas mass spectrometer detector is very expensive, which greatly increases the production cost.

Thus, it is desirable to provide bioreactors, which can overcome one or more of the above defects.

### SUMMARY

According to an aspect, a bioreactor is provided. The bioreactor may include a tank configured to contain a mixture of animal cells and a liquid; and a first ventilation device arranged outside the tank and including a first gas distributor, the first gas distributor being configured to transmit a gas to the liquid separated from the animal cells.

In some embodiments, the liquid includes a culture medium; and the first gas distributor includes a shell and a first diaphragm arranged in the shell, the shell of the first gas distributor includes a hollow inner cavity, and the first diaphragm separates the hollow inner cavity of the shell into a culture medium chamber through which the culture medium flows and a gas chamber through which the gas flows, the culture medium chamber of the first gas distributor is in fluid communication with the tank through a liquid outlet pipe, and the first diaphragm dissolves the gas in the gas chamber into the culture medium in the culture medium chamber without bubbles.

In some embodiments, the first diaphragm is arranged to make the culture medium chamber and the gas chamber in a form of an inner cylinder and an outer cylinder nested with each other.

In some embodiments, the gas chamber includes a first ventilation slot arranged along an inner surface of a sidewall of the shell, the first ventilation slot being configured to guide an inlet gas to flow on the entire inner surface of the sidewall; the first diaphragm is arranged on the inner surface of the sidewall and covers the first ventilation slot.

In some embodiments, the sidewall comprises a plurality of convex parts, and the plurality of convex parts protrude radially inward from the inner surface of the sidewall; and the first ventilation slot is formed in a space between the inner surface of the sidewall and side surfaces of adjacent convex parts.

In some embodiments, the first diaphragm is in a form of a dense membrane or a microporous membrane, a pore size of the microporous membrane ventilates the first diaphragm without bubbles under a certain pressure, and a thickness of the dense membrane is between 50 μm to 500 μm.

In some embodiments, the first ventilation device further includes a first gas inlet unit, which is in fluid communication with the gas chamber through a gas inlet pipe and transmits the inlet gas to the gas chamber, and a first gas outlet unit, which is in fluid communication with the gas chamber through a gas outlet pipe and discharges an undissolved gas in the gas chamber from the first gas distributor.

In some embodiments, the first gas inlet unit includes a first gas flow component regulator arranged on the gas inlet pipe, the first gas flow component regulator being configured to adjust a component proportion of the inlet gas; and the first gas outlet unit includes a pressure control valve and a sensor arranged in the gas outlet pipe to control gas pressure in the gas chamber.

In some embodiments, the first ventilation device further includes a first dissolved oxygen electrode arranged in the mixture, the first dissolved oxygen electrode detects a dissolved oxygen concentration value in the mixture; and a first controller, the first controller adjusting a proportion of oxygen in the inlet gas of the first gas flow component regulator through the dissolved oxygen concentration value detected by the first dissolved oxygen electrode, so as to adjust the dissolved oxygen concentration value of the mixture in the tank.

In some embodiments, the first ventilation device further includes a first pH electrode arranged in the mixture, the first pH electrode being configured to detect a pH value in the mixture; and the first controller is configured to adjust a proportion of carbon dioxide in the inlet gas of the second gas flow component regulator through a pH value detected by the first pH electrode, so as to adjust the pH value of the mixture.

In some embodiments, the bioreactor further includes a dialysis component, which is arranged outside the tank and comprises a dialysis filter, wherein the dialysis filter is configured to dialyze harmful metabolites in the culture medium separated from the animal cells into a dialysate; and the dialysis component is in fluid communication with the first ventilation device, and the first gas distributor and the dialysis filter are formed as an integral part.

In some embodiments, the dialysis filter comprises a shell, a filter element arranged in the shell, and a hollow inner cavity, the shell of the dialysis filter is in a shape of a cylinder with a top wall, a bottom wall and a sidewall extending between the top wall and the bottom wall, and the filter element separates the hollow inner cavity of the shell of the dialysis filter into a culture medium chamber through which the culture medium flows and a dialysate chamber through which the dialysate flows; and the culture medium chamber of the dialysis filter and a culture medium chamber of the first gas distributor are in fluid communication through a connecting pipe.

In some embodiments, the connecting pipe and at least one of a corresponding part of the shell of the dialysis filter or a corresponding part of the shell of the first gas distributor are arranged to be transparent so that a flow state of the culture medium in the connecting pipe can be observed.

In some embodiments, the culture medium chamber of the dialysis filter is in fluid communication with the tank through a liquid inlet pipe; the dialysis component further comprises a fresh dialysate storage tank and a waste dialysate storage tank, both of which are in fluid communication with the dialysate chamber; the dialysis filter is arranged on the pipe between the dialysate chamber and the fresh dialysate storage tank for filtering out insoluble particles in the dialysate.

In some embodiments, the bioreactor further includes a cell separation device configured to separate the culture medium from the animal cells, and the cell separation device is arranged in the tank and is in fluid communication with the dialysis filter.

In some embodiments, the bioreactor further includes a second ventilation device arranged in the tank and configured to transmit the gas to the mixture in the tank.

In some embodiments, the second ventilation device includes a second gas distributor, the second gas distributor is configured to be immersed in the mixture of the tank and includes a distributor body and a second diaphragm supported by the distributor body, wherein the distributor body is provided with a second ventilation slot, and the second diaphragm is configured to dissolve the gas in the second ventilation slot into the mixture without bubbles.

In some embodiments, the second ventilation device includes a second gas inlet unit, which is in fluid communication with the second ventilation slot and transmits the inlet gas to the second ventilation slot, and a second gas outlet unit, which is in fluid communication with the second ventilation slot and discharges an undissolved gas in the second ventilation slot from the distributor body.

In some embodiments, the second gas inlet unit is in fluid communication with the second ventilation slot through a gas inlet pipeline, the second gas outlet unit is in fluid communication with the second ventilation slot through a gas outlet pipeline; and the second gas inlet unit includes a second gas flow component regulator arranged on the gas inlet pipeline and electrically connected to a second controller, the second gas flow component regulator is configured to maintain a total flow of the inlet gas at a constant value and adjust a component proportion of the inlet gas according to a command of the second controller.

In some embodiments, the second controller is configured to monitor and adjust an oxygen uptake rate OUR consumed by the animal cells in the tank in real-time, the oxygen uptake rate OUR is determined by a calculation formula of OUR=(Cout−Csensor)*KLa, where Cout denotes a dissolved oxygen concentration value detected by a second dissolved oxygen electrode which is arranged in a gas outlet pipeline; Csensor denotes a dissolved oxygen concentration value detected by a third dissolved oxygen electrode arranged in the mixture; and KLA is a mass transfer coefficient of the second gas distributor, wherein the mass transfer coefficient of the second gas distributor is related to a rotational speed of a motor in the tank, the motor driving the circulating flow of the mixture.

### BRIEF DESCRIPTION OF THE DRAWINGS

After reading the following specific embodiments in combination with the drawings, various aspects of the present disclosure will be better understood. In the drawings:

FIG. 1 is a schematic diagram illustrating an exemplary bioreactor according to some embodiments of the present disclosure;

FIG. 2A is a perspective diagram illustrating a part of the bioreactor shown in FIG. 1; FIG. 2B is an upper cross-sectional diagram illustrating a part of the bioreactor shown in FIG. 1; FIG. 2C is a lower cross-sectional diagram illustrating a part of the bioreactor shown in FIG. 1;

FIGS. 3A and 3B are schematic diagrams illustrating exemplary diaphragms of the bioreactor shown in FIG. 1;

FIGS. 4A and 4B illustrates an assembled perspective view and a cross-sectional view of a gas distributor in another example of the bioreactor shown in FIG. 1, respectively; FIG. 4C illustrates a partial stereoscopic view of a diaphragm of the gas distributor; and

FIG. 5 is a schematic diagram illustrating an exemplary bioreactor according to some other embodiments of the present disclosure;

FIG. 6 is a schematic diagram illustrating an exemplary bioreactor according to some other embodiments of the present disclosure;

FIG. 7 is an exploded perspective diagram of a gas distributor of the ventilation device shown in FIG. 6; and

FIG. 8 is a schematic diagram illustrating an exemplary relationship between a mass transfer coefficient of the diaphragm, a motor speed and a total ventilation volume.

### DETAILED DESCRIPTION

The present disclosure will be described below with reference to the drawings, which show several embodiments of the present disclosure. However, it should be understood that the present disclosure may be presented in a variety of different ways and is not limited to the embodiments described below. In fact, the embodiments described below are intended to make the disclosure of the present disclosure more complete and fully explain the scope of protection of the present disclosure to those skilled in the art. It should also be understood that the embodiments disclosed in the present disclosure can be combined in various ways to provide more additional embodiments.

It should be understood that in the drawings corresponding to the same embodiment, the same reference numerals refer to the same or similar elements, but in the drawings corresponding to different embodiments, the same reference numerals may refer to different elements. In the drawings, the dimensions of some features can be deformed for clarity.

It should be understood that the use of words in the specification are only used to describe specific embodiments and are not intended to limit the present disclosure. Unless otherwise defined, all terms (including technical terms and scientific terms) used in the present disclosure have meanings commonly understood by those skilled in the art. Well-known functions or constructions may not be described in detail for brevity and/or clarity.

As shown in the present disclosure and claim, unless the context clearly prompts the exception “a”, “an”, “this”, and/or “the” is not specifically singular, and the plural may be included. The terms “comprises,” “comprising,” “includes,” “including” and/or “contain” used in the present disclosure indicate the existence of the claimed features, but do not exclude the existence of one or more other features. The term “and/or” used in the present disclosure includes any and all combinations of one or more of the relevant listed items. The terms “between X and Y” and “between about X and Y” used in the present disclosure shall be interpreted to include X and Y. The term “between about X and Y” used in the present disclosure means “between about X and about y”, and the term “from about X to Y” used in the present disclosure means “from about X to about Y”.

In the present disclosure, when an element is said to be “on”, “attached” to another element, “connected” to another element, “coupled” to another element, or “in contact” with another element, the element can be directly on another element, attached to another element, connected to another element, or in contact with another element, or there can be an intermediate element. In contrast, when an element is said to be “directly” on another element, “directly attached” to another element, “directly connected” to another element, “directly coupled” to another element, or “directly in contact” with another element, there will be no intermediate element. In the present disclosure, one feature is arranged to be “adjacent” to another feature, which refers to one feature that has a part overlapping with the adjacent feature or a part above or below the adjacent feature.

In the present disclosure, the spatial relationship terms such as “up”, “down”, “left”, “right”, “front”, “back”, “above”, “below” and so on can explain the relationship between one feature and another feature in the drawings. It should be understood that the spatial relationship term includes different orientations of the device in use or operation in addition to the orientations shown in the drawings. For example, when the device in the drawings is reversed, the feature originally described as “below” other features can be described as “above” other features. The device can also be oriented in other ways (rotated 90 degrees or in other directions), and the relative spatial relationship will be explained accordingly.

A bioreactor is a vessel-like device that provides a uniform background for microorganisms to grow and maintains an uninterrupted balance in the biochemical reactions carried out by these microorganisms to produce desired metabolites. The bioreactor may be applied for biomass production such as single cell protein, baker's yeast, animal cells, and microalgae. In some embodiments, the bioreactor may be a small bioreactor, e.g., a disposable bioreactor. In some embodiments, the bioreactor may be a bioreactor for animal cells.

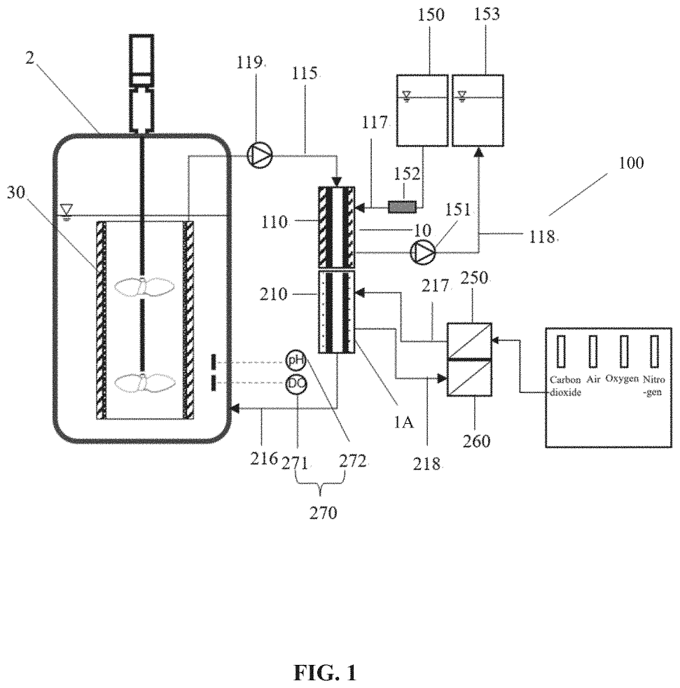

FIG. 1 is a schematic diagram illustrating an exemplary bioreactor according to some embodiments of the present disclosure. The bioreactor 100 includes the tank 2 and a first ventilation device 1A arranged outside the tank 2. The tank 2 may be cylindrical or any other suitable shape, and made of, e.g., stainless steel, plastic, etc. The first ventilation device 1A is used to transmit one or more gases (such as a mixture of oxygen, carbon dioxide, and air, or a mixture of oxygen, carbon dioxide, and nitrogen) to the liquid (e.g., the culture medium) separated from animal cells outside the tank 2. In some embodiments, the bioreactor 100 further includes a dialysis component 10 in fluid communication with each other. The dialysis component 10 is used to dialyze harmful metabolites (such as lactic acid, inorganic ammonium, etc.) in the culture medium outside the tank 2. The combination of the dialysis component and the first ventilation device herein may be referred to as an external circulation renewal device of a culture medium. In some embodiments, the bioreactor 100 also optionally includes a cell separation device 30 in any form. The cell separation device 30 is arranged in the tank 2 and is used to separate the liquid (e.g., culture medium) from the animal cells in the tank 2, thereby supplying the liquid (e.g., the culture medium) separated from the animal cells to the dialysis component 10, so as to eliminate shear damage of a supply circulating pump to the animal cells.

Hereinafter, taking the liquid being the culture medium to describe the components. The dialysis component 10 includes a dialysis filter 110. The dialysis filter 110 is used to dialyze harmful metabolites in the liquid (e.g., the culture medium) into the dialysate. The dialysis filter 110 includes a shell 111 and a filter element 112 arranged in the shell 111. The shell 111 is a shape of a cylinder and includes a top wall 111T, a bottom wall 111B, and a sidewall 111S extending between the top wall 111T and the bottom wall 111B. The top wall 111T, the bottom wall 111B, and the sidewall 111S surround a hollow inner cavity of the shell 111. The cross-section of the shell 111 may be circular, elliptical, triangular, quadrilateral, or any other shape.

The filter element 112 separates the hollow inner cavity of the shell 111 into a culture medium chamber 113 and a dialysate chamber 114. The culture medium chamber 113 is used for the flow of the culture medium, and the dialysate chamber 114 is used for the flow of the dialysate so that the harmful metabolites in the culture medium enter the dialysate of the dialysate chamber 114 from the culture medium chamber 113 through the filter element 112. The filter element 112 may be in a shape of a cylinder with both ends open, the top of the filter element 112 is fixedly connected to the top wall 111T of the shell 111 by means of glue, hot melt, or ultrasonic, the bottom of the filter element 112 is fixedly connected to the bottom wall 111B of the shell 111 by means of glue, hot melt, or ultrasonic. Thus, the filter element 112 separates the hollow inner cavity of the shell 111 into the culture medium chamber 113 and the dialysate chamber 114 in a form of an inner cylinder and an outer cylinder nested with each other, respectively.

The medium chamber 113 is provided with a liquid inlet 113I and a liquid outlet 113O (for example, on the top wall 111T and the bottom wall 111B of the shell 111). The liquid inlet 113I is in fluid communication with the cell separation device 30 through a liquid inlet pipe 115 to receive the culture medium separated from the animal cells from the cell separation device 30. A pump 119 (e.g., a peristaltic pump) may be provided on the liquid inlet pipe 115 to pump the culture medium separated from the animal cells from the cell separation device 30 into the culture medium chamber 113. The cell separation device 30 is used to separate the culture medium from the animal cells so that the culture medium passing through the liquid inlet pipe 115 does not contain the animal cells, which avoids shear damage to animal cells caused by the pump 119. The liquid outlet 113O is in fluid communication with the first ventilation device 1A through a connecting pipe 116 to deliver the dialyzed medium to the first ventilation device 1A. The connecting pipe 116 is roughly funnel-shaped, and one end is connected to the liquid outlet 113O and the other end is connected to a liquid inlet 213I of a culture medium chamber 213 of the first ventilation device 1A (which will be described in detail below). In some embodiments, the connecting pipe 116 and its corresponding shell parts on the outside are arranged to be transparent, so that the flow state of the culture medium in the connecting pipe 116 can be observed. The dialysate chamber 114 is provided with a liquid inlet 114I and a liquid outlet 114O (for example, on the sidewall 111S). The liquid inlet 114I is in fluid communication with a fresh dialysate storage tank 150 through a liquid inlet pipe 117, and the liquid outlet 114O is in fluid communication with a waste dialysate storage tank 153 through a liquid outlet pipe 118.

In other embodiments, the filter element 112 may be in any other shape. For example, the filter element 112 may be in a sheet shape, and the top of the filter element 112 is fixedly connected to the top wall 111T of the shell 111, while the bottom is fixedly connected to the bottom wall 111B of the shell 111. Thus, the filter element 112 separates the hollow inner cavity of the shell 111 into the culture medium chamber 113 and the dialysate chamber 114 in the form of a half cylinder adjacent to each other.

The fresh dialysate storage tank 150 is used to store fresh dialysate and is in fluid communication with the dialysate chamber 114 of the shell 111 through the liquid inlet pipe 117. The waste dialysate storage tank 153 is used to store waste dialysate and is in fluid communication with the dialysate chamber 114 of the shell 111 through the liquid outlet pipe 118. A pump 151 (for example, a peristaltic pump) may be arranged on the liquid inlet pipe 117 and/or the liquid outlet pipe 118 to drive the dialysate to flow between the fresh dialysate storage tank 150, the dialysate chamber 114, and the waste dialysate storage tank 153. That is, the fresh dialysate is pumped from the fresh dialysate storage tank 150 to the dialysate chamber 114 through the liquid inlet pipe 117, the used waste dialysate is pumped from the dialysate chamber 114 to the waste dialysate storage tank 153 through the liquid outlet pipe 118. A filter 152 is arranged on the liquid inlet pipe 117 to filter out insoluble particles in the dialysate. A controller (not shown, which is a same or different controller as the first controller) may be electrically connected to the pump 151 and a pump 119 to control the operation of the pumps.

In some embodiments, the first ventilation device 1A includes a first gas distributor 210 as well as a first gas inlet unit 250 and a first gas outlet unit 260 which are in fluid communication with the first gas distributor 210. The first gas inlet unit 250 is used to deliver a gas to the first gas distributor 210. The first gas distributor 210 is used to dissolve the gas received from the first gas inlet unit 250 into the culture medium. The first gas outlet unit 260 is used to exhaust a remaining gas in the first gas distributor 210 that is not dissolved in the culture medium from the first gas distributor 210. The first ventilation device 1A may also include a first solution monitoring unit 270 to monitor various parameters of the mixture of animal cells and culture medium in the tank 2, such as a dissolved oxygen concentration value and a pH value. A first controller (not shown) may be electrically connected to the first gas inlet unit 250, the first gas outlet unit 260, and the first solution monitoring unit 270 to control the gas inlet and gas outlet operations.

The first gas distributor 210 and the dialysis filter 110 are formed as an integral part. The first gas distributor 210 includes a shell 211 and a first diaphragm 212 disposed in the shell 211. The shell 211 accommodates the dialyzed culture medium received from the culture medium chamber 113 of the dialysis component 10 and the gas to be dissolved into the culture medium received from the first gas inlet unit 250. The first diaphragm 212 transmits and dissolves the gas on one side of the first diaphragm 212 into the culture medium on the other side of the first diaphragm 212 without bubbles in the shell 211. The shell 211 is in a shape of a cylinder with a top wall 211T, a bottom wall 211B, and a sidewall 211S extending between the top wall 211T and the bottom wall 211B. The top wall 211T, the bottom wall 211B, and the sidewall 211S surround the hollow inner cavity of the shell 211. The cross-section of the shell 211 may be circular, elliptical, triangular, quadrilateral, or any other shape. The cross-section of the shell 211 may be set to correspond to the cross-section of the shell 111 of the dialysis filter 110, or not correspond to the cross-section of the shell 111 of the dialysis filter 110. The sidewall 211S of the shell 211 may extend upward over the top wall 211T of the shell 211, and/or the sidewall 111S of the shell 111 can extend down over the bottom wall 111B of the shell 111, so that the sidewall 211S of the shell 211 and the sidewall 111S of the shell 111 may be formed integrally, or may be formed separately and fixed together by welding (e.g., ultrasonic welding), bonding, snap connection, etc.

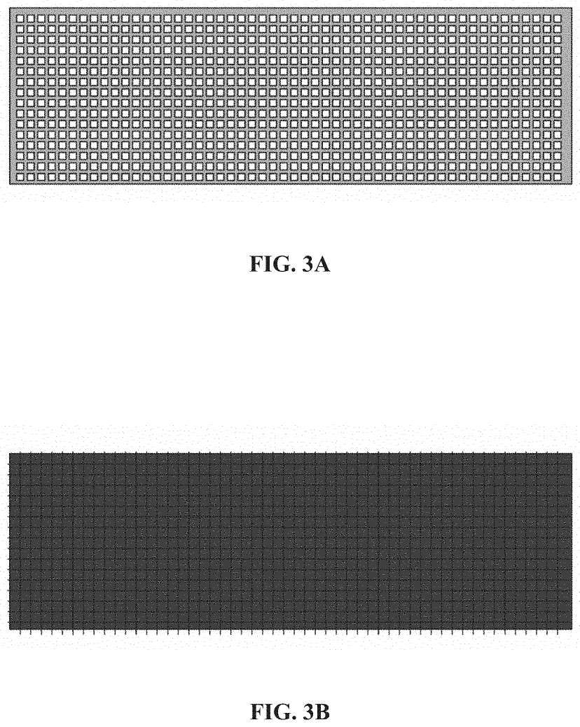

The first diaphragm 212 separates the hollow inner cavity of the shell 211 into the culture medium chamber 213 and a gas chamber 214. The culture medium chamber 213 is used for the culture medium to flow through, and the gas chamber 214 is used for the gas to flow through. Therefore, the gas is dissolved into the culture medium of the culture medium chamber 213 in a bubble-free manner by passing through the first diaphragm 212 from the gas chamber 214. The first diaphragm 212 may be made of silica gel, polydimethylsiloxane (PDMS), polycarbonate track-etch (PCTE), polyethylene terephthalate (PETE), polytetrafluoroethylene (PTFE), polypropylene (PP), polycarbonate (PC), nylon, polyethersulfone (PES), sintered porous material, or the like. The first diaphragm 212 may be processed with hydrophilic and positive charge or negative charge treatment so that it is not easy to be blocked due to being adsorbed by animal cells. The first diaphragm 212 may be in the form of a microporous membrane (see FIG. 3A). The microporous membrane may adopt different pore sizes according to different biological processes (such as stem cells, tumor cells, CHO cells, or microcarrier processes), and the pore sizes are less than 0.05 μm, for example, including but not limited to 0.01 μm, 0.05 um, 0.10 μm, 0.20 μm, 0.04 μm, etc. With the above-mentioned pore sizes, the first diaphragm 212 does not produce bubbles when ventilated under a certain pressure. In addition, the first diaphragm 212 may be in the form of a dense membrane (see FIG. 3B). The dense membrane may be made of, for example, a silica gel material of PDMS. The dense membrane has no concept of pore sizes, but it is required to be as thin as possible, such as 50 μm to 500 μm.

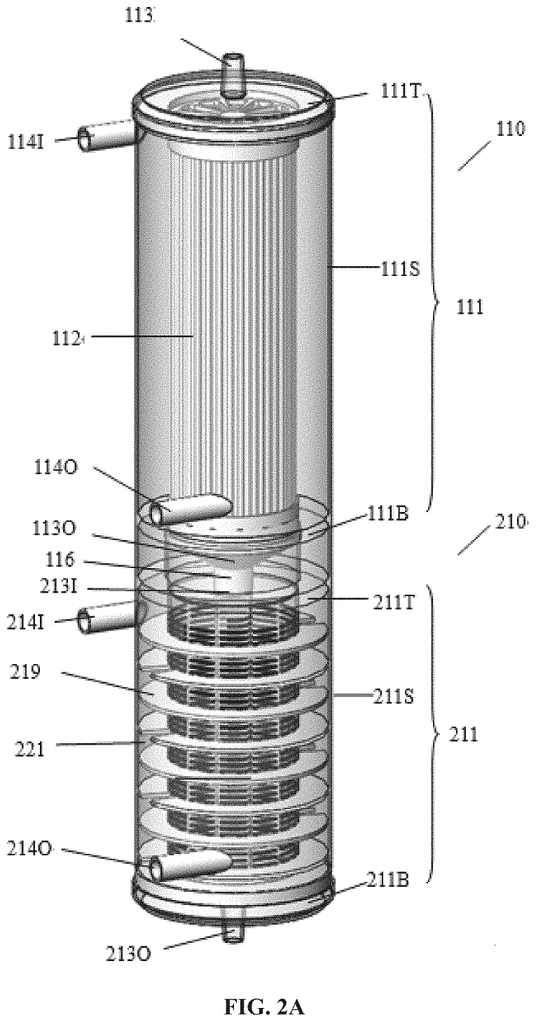

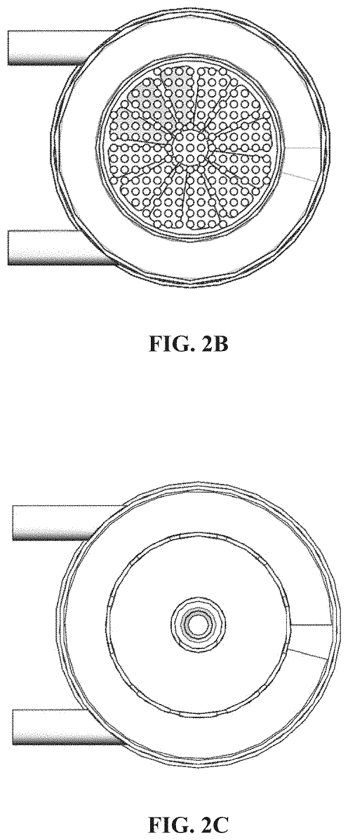

As shown in FIG. 2A, the gas chamber 214 may include a first ventilation slot 221 extending on the sidewall 211S of the shell 211. The plurality of convex parts 219 protrude radially inward from the inner surface of the sidewall 211S, and the first ventilation slot 221 is formed in a space between the inner surface of the sidewall 211S and the side surface of the adjacent convex parts 219. The first ventilation slot 221 is used to guide the flow of gas on the entire inner surface of the sidewall 211S to increase the residence time of the gas in the first gas distributor 210 and promote gas mass transfer.

The convex parts 219 may form a support frame for supporting the first diaphragm 212, thereby keeping the first diaphragm 212 flat. The first diaphragm 212 may be fixed to the radial inner surfaces of the convex parts 219 of the sidewall 211S by bonding or other means, and may cover the first ventilation slot 221.

The first ventilation slot 221 is in fluid communication with the first gas inlet unit 250 through a gas inlet 214I on the sidewall 211S of the shell 211 and with the first gas outlet unit 260 through a gas outlet 214O on the sidewall 211S of the shell 211, thereby forming a gas flow channel through the gas inlet 211, the first ventilation slot 221 and the gas outlet 214O on the shell 211. The gas may flow into the first ventilation slot 221 from the gas inlet 214I, dissolve into the culture medium of the culture medium chamber 213 through the first diaphragm 212 above the first ventilation slot 221, and the undissolved gas flows out of the first ventilation slot 221 through the gas outlet 214O.

The first ventilation slot 221 may be in a serpentine shape winding mainly along a horizontal direction on the entire inner surface of the sidewall 211S. The first ventilation slot 221 may also be arranged on the sidewall 211S in various other patterns. For example, in one embodiment, the first ventilation slot 221 may be in a serpentine shape winding mainly along a vertical direction on the entire inner surface of the sidewall 211S.

In another embodiment, the first ventilation slot 221 may be in a spiral shape extending from the bottom to the top around the entire inner surface of the sidewall 211S.

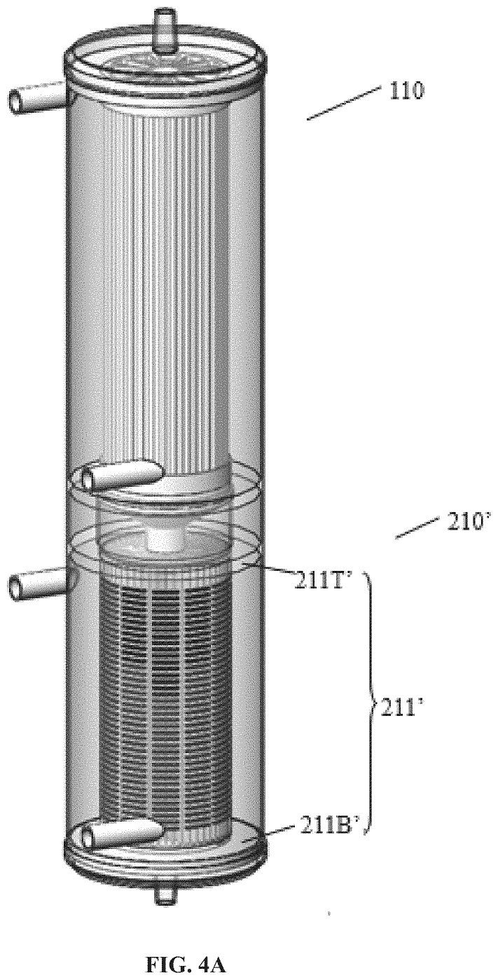

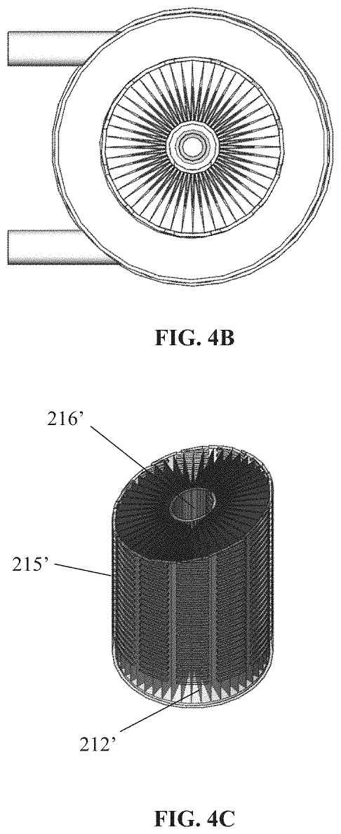

FIG.4A and FIG.4B respectively illustrate an assembled perspective diagram and a cross-sectional diagram of another form of a gas chamber 214′ and an associated first gas distributor 210′, and FIG. 4C shows a perspective diagram of a first diaphragm 212′ of the first gas distributor 210′ (the upper half is removed to see the internal structure). As shown in the figures, the first diaphragm 212′ may be formed into a cylinder with both ends open, and a sidewall of the cylinder may be serrated or wavy. The top of the first diaphragm 212′ is fixedly connected to a top wall 211T′ of the shell 211′ by means of glue, hot melt, or ultrasonic, while the bottom is fixedly connected to a bottom wall 211B′ of the shell 211′ by means of glue, hot melt, or ultrasonic. Thus, the first diaphragm 212′ separates the hollow inner cavity of the shell 211 into a medium chamber 213′ and a gas chamber 214′ in the form of an inner cylinder and an outer cylinder nested with each other. Non-woven fabric may be bonded to the entire surface of the first diaphragm 212′ by bonding, welding, etc., so as to enhance the strength of the first diaphragm 212′ in the circumferential and vertical directions of the cylinder of the first diaphragm, thereby preventing the collapse of the first diaphragm 212′. In some embodiments, cylindrical supports 215′ and 216′ may be respectively added to a radial inner side and an outer side of the first diaphragm 212′ to support the first diaphragm 212′.

Return to FIG. 1 and FIG. 2A, the culture medium chamber 213 is provided with the liquid inlet 213I and the liquid outlet 213O, respectively (for example, on the top wall 211T and the bottom wall 211B of the shell 211). The liquid inlet 213I is in fluid communication with the culture medium chamber 113 of the dialysis component 10 through the connecting pipe 116 to receive the dialyzed culture medium from the dialysis component 10. The liquid outlet 213O is in fluid communication with the tank 2 through a liquid outlet pipe 216 to transport the culture medium after dialysis treatment and gas transmission back to the tank 2. The gas chamber 214 is provided with the gas inlet 214I and the gas outlet 214O (for example, on the top wall 211T, the bottom wall 211B, or the sidewall 211S). The gas inlet 214I is in fluid communication with the first gas inlet unit 250 through the gas inlet pipe 217, and the gas outlet 214O is in fluid communication with the first gas outlet unit 260 through the gas outlet pipe 218 to allow the gas to flow through the gas chamber 214.

The first gas inlet unit 250 is connected to the gas inlet 214I through the gas inlet pipe 217 and is arranged outside the first gas distributor 210. The first gas inlet unit 250 transmits the gas from the outside to the first ventilation slot 221 in a controlled manner. The first gas inlet unit 250 includes a first gas flow component regulator (such as a mass flowmeter or a gas-proportion regulating valve) arranged on the gas inlet pipe 217. The first gas flow component regulator regulates the flow and component proportion of the inlet gas. In this embodiment, the inlet gas may be mainly composed of air, oxygen, and carbon dioxide, or mainly composed of nitrogen, oxygen, and carbon dioxide. The first gas flow component regulator is controlled by a first controller to adjust the proportion of oxygen and carbon dioxide in the total flow in real-time, so as to adjust the dissolved oxygen concentration and the pH value in the mixture.

The first gas outlet unit 260 is connected to the gas outlet 214O through the gas outlet pipe 218 and is arranged outside the first gas distributor 210. The first gas outlet unit 260 discharges the undissolved gas in the first ventilation slot 221 in a controlled manner to prevent the pressure increase in the first gas distributor 210 from affecting the gas control of the first gas inlet unit 250. The first gas outlet unit 260 includes a pressure control valve and a sensor arranged in the gas outlet pipe 218. The pressure control valve and sensor are controlled by the first controller to adjust the gas pressure (generally maintained between 0.01 Mpa-0.1 Mpa) in the first ventilation slot 221 of the first gas distributor 210, so as to maintain the mass transfer efficiency of the first diaphragm 212.

The first solution monitoring unit 270 includes a first dissolved oxygen electrode 271 and a first pH electrode 272 electrically connected to the first controller. The first dissolved oxygen electrode 271 and the first pH electrode 272 may be arranged in a mixture of the cell and the culture medium (or other liquids) in the tank 2. The first dissolved oxygen electrode 271 is used to detect the dissolved oxygen concentration value in the mixture and feed it back to the first controller. The first pH electrode 272 is used for determining the pH value of the mixture and fed back to the first controller. The first controller adjusts the oxygen proportion of the inlet gas in the first gas flow component regulator of the first gas inlet unit 250 in real-time through the dissolved oxygen concentration value detected by the first dissolved oxygen electrode 271 of the first solution monitoring unit 270. For example, if the dissolved oxygen of the mixture is set to no less than 40% during the culture process, with the increase of the number of animal cells in the mixture, the oxygen flow also needs to be increased, and the oxygen proportion in the total gas flow also increases in order to maintain the same dissolved oxygen. In addition, the first controller adjusts the proportion of carbon dioxide of the inlet gas in the first gas flow component regulator of the first gas inlet unit 250 through the pH value detected by the first pH electrode 272 of the first solution monitoring unit 270, so as to adjust the pH value of the mixture. For example, if the pH value of the mixture is set to a constant value during the culture process, with the continuous increase of pH during the culture process, the flow of carbon dioxide also needs to be increased, and the proportion of carbon dioxide in the total gas flow also increases in order to maintain a constant pH value.

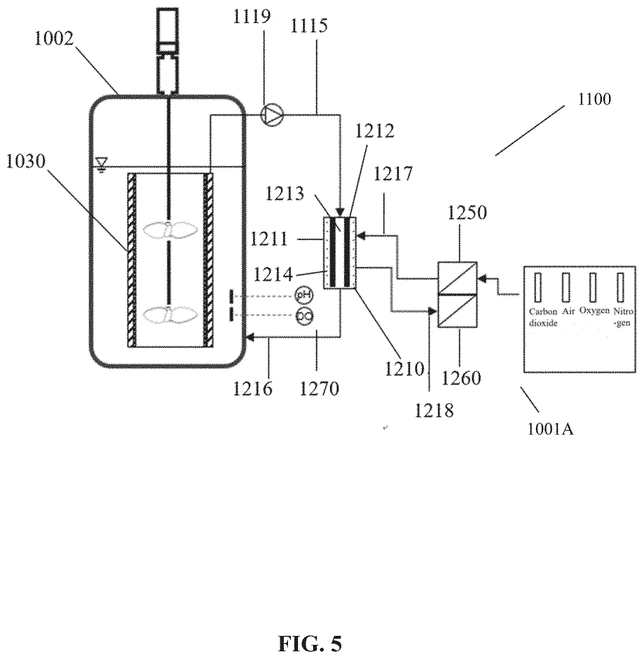

FIG. 5 is a schematic diagram illustrating an exemplary bioreactor 1100 according to some other embodiments of the present disclosure. For the bioreactor 1100, the reference symbols of the bioreactor 100 plus 1000 represent the same or similar structure.

The bioreactor 1100 may be a small bioreactor (such as a disposable animal cell bioreactor). The bioreactor 1100 includes a tank 1002. The tank 1002 is used to accommodate a mixture of animal cells and a liquid (e.g., a culture medium). The bioreactor 1100 has the function of renewing a culture medium separated from animal cells outside the tank 1002, such as transmitting one or more gases to the culture medium. The main difference between the bioreactor 1100 and the bioreactor 100 is that the bioreactor 1100 does not include a dialysis component.

As shown in FIG. 5, the bioreactor 1100 may include a first ventilation device 1001B. The first ventilation device 1001B is used to transmit one or more gases (such as a mixture of oxygen, carbon dioxide, and air, or a mixture of oxygen, carbon dioxide, and nitrogen) to the liquid (e.g., the culture medium) outside the tank 1002. In some embodiments, the bioreactor 1100 also optionally includes a cell separation device 1030 in any form. The cell separation device 1030 is arranged in the tank 1002 and is used to separate the liquid (e.g., culture medium) from the animal cells in the tank 1002 to supply the culture medium separated from the animal cells to the first ventilation device 1001A, so as to eliminate the shear damage of the supply circulating pump to the animal cells.

The first ventilation device 1001A includes a first gas distributor 1210, a first gas inlet unit 1250 in fluid communication with the first gas distributor 1210, and a first gas outlet unit 1260 in fluid communication with the first gas distributor 1210. The first gas inlet unit 1250 is used to transmit gas to the first gas distributor 1210. The first gas distributor 1210 is used to dissolve the gas received from the first gas inlet unit 1250 into the liquid (e.g., the culture medium). The first gas outlet unit 1260 is used to discharge the remaining gas in the first gas distributor 210 that is not dissolved in the culture medium from the first gas distributor 1210. The first ventilation device 1001A may also include a first solution monitoring unit 1270 to monitor various parameters of the mixture of animal cells and the liquid (e.g., the culture medium) in the tank 1002, such as the dissolved oxygen concentration value and the pH value. A controller (not shown, similar to the first controller) may be electrically connected to the first gas inlet unit 1250, the first gas outlet unit 1260, and the first solution monitoring unit 1270 to control the gas inlet and outlet operations.

The first gas distributor 1210 includes a shell 1211 and a first diaphragm 1212 disposed in the shell 1211. The shell 1211 accommodates the liquid (e.g., the culture medium) separated from animal cells received from the cell separation device 1030 and the gas to be dissolved into the culture medium received from the first gas inlet unit 1250. The first diaphragm 1212 transmits and dissolves the gas on one side of the shell 1211 into the culture medium on the other side without bubbles.

The first diaphragm 1212 separates the hollow inner cavity of the shell 1211 into a culture medium chamber 1213 and a gas chamber 1214. The culture medium chamber 1213 is used for the culture medium to flow through, and the gas chamber 1214 is used for the gas to flow through. Therefore, the gas passes through the first diaphragm 1212 from the gas chamber 1214 and is dissolved into the culture medium of the culture medium chamber 1213 in a bubble-free manner.

A liquid inlet of the culture medium chamber 1213 is in fluid communication with the cell separation device 1030 through a connecting pipe 1115 to receive the culture medium separated from animal cells from the cell separation device 1030. A pump 1119 (e.g., a peristaltic pump) may be arranged on the liquid inlet pipe 1115 to pump the culture medium separated from animal cells from the cell separation device 1030 to the culture medium chamber 1213. The cell separation device 1030 is used to separate the culture medium from animal cells, so that the culture medium passing through the liquid inlet pipe 1115 does not contain animal cells, which avoids shear damage to animal cells caused by the pump 1119. A liquid outlet of the medium chamber 1213 is in fluid communication with the tank 1002 through a liquid outlet pipe 1216 to transport the gas-transported medium back to the tank 1002. The gas inlet of the gas chamber 1214 is in fluid communication with the first gas inlet unit 1250 through a gas inlet pipe 1217, and the gas outlet is in fluid communication with the first gas outlet unit 1260 through a gas outlet pipe 1218 to make the gas flow through the gas chamber 1214.

The structure and function of the first gas distributor 1210 are similar to the structure and function of the first gas distributor 210 or similar to the structure and function of the first gas distributor 210′. The structures and functions of the first gas inlet unit 1250, the first gas outlet unit 1260 and the first solution monitoring unit 1270 are similar to the structures and functions of the first gas inlet unit 250, the first gas outlet unit 260, and the first solution monitoring unit 270. Therefore, the structures and functions of the first gas distributor 1210, the first gas inlet unit 1250, the first gas outlet unit 1260 and the first solution monitoring unit 1270 may be understood with reference to the embodiments in FIGS. 5-7.

Based on the cell separation device in the tank, the culture medium separated from the animal cells is circulated and renewed, and the shear damage to the animal cells by the circulating pump is reduced.

The bioreactor 100 according to the present disclosure integrates the gas exchange function and the dialysis function into a whole, has a compact design, reduces the volume of the device, and reduces the production cost.

The bioreactor (e.g., the bioreactors 100 and 1100) according to the present disclosure uses a microporous membrane or a dense membrane to dissolve the gas, which is more conducive to the transmission effect of the gas. The external circulation renewal device of the culture medium according to the present disclosure does not generate phenomena such as lots of foam, uneven oxygen transmission, and limited oxygen transmission.

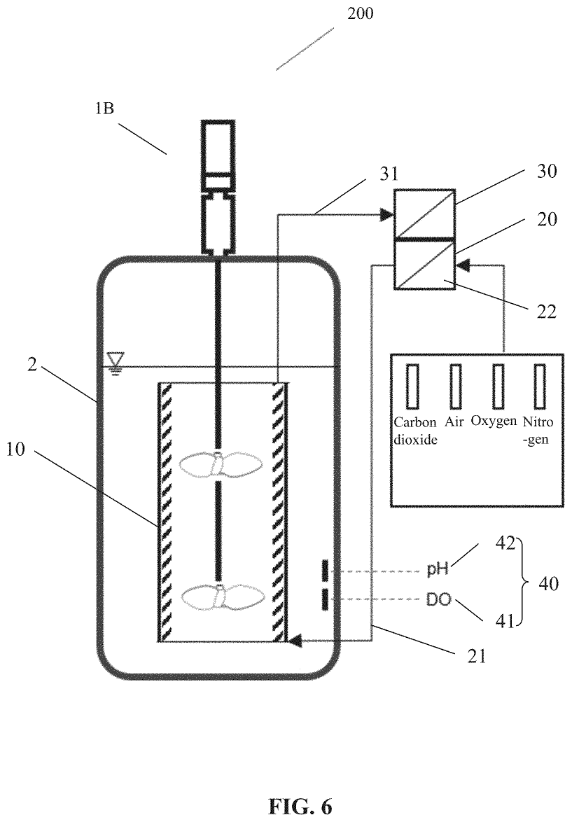

FIG. 6 is a schematic diagram illustrating an exemplary bioreactor according to some embodiments of the present disclosure. The bioreactor 200 includes a tank 2 and a second ventilation device 1B arranged in the tank 2. The tank 2 is configured to contain a mixture of animal cells and a liquid (e.g., a culture medium, water). The second ventilation device 1B is used to transmit one or more gases, such as a mixture of oxygen, carbon dioxide, and air, or a mixture of oxygen, carbon dioxide, and nitrogen, to the mixture of animal cells and the liquid (e.g., culture medium) in the tank 2. As shown in the figure, the second ventilation device 1B may include a second gas distributor 10, as well as a second gas inlet unit 20 and a second gas outlet unit 30 which are in fluid communication with the second gas distributor 10. The second gas inlet unit 20 is used to transmit a gas to the second gas distributor 10. The second gas distributor 10 is used to dissolve the gas received from the second gas inlet unit 20 into the mixture of animal cells and liquid in the tank 2. The second gas outlet unit 30 is used to discharge a remaining gas in the second gas distributor 10 that is not dissolved in the mixture from the second gas distributor 10. The second ventilation device 1B may also include a second solution monitoring unit 40 to monitor various parameters of the mixture in the tank 2, such as a dissolved oxygen concentration and/or a pH value. The second ventilation device 1B may also include a controller (not shown, which is the same or different controller as the second controller) electrically connected to the second gas inlet unit 20, the second gas outlet unit 30, and the second solution monitoring unit 40.

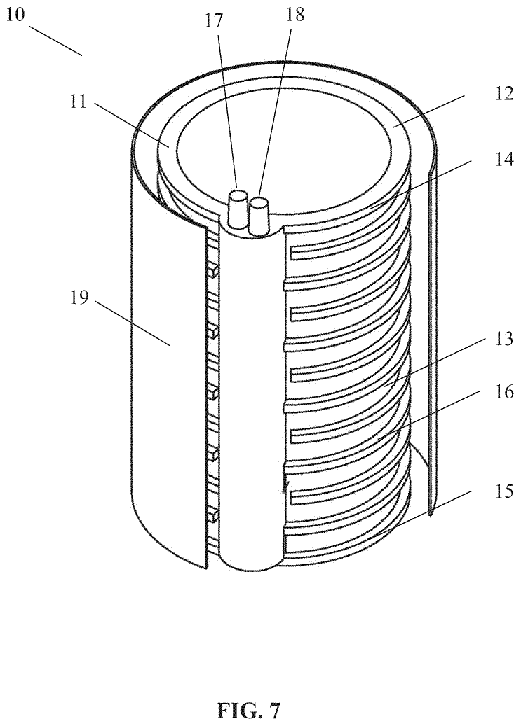

The second gas distributor 10 is immersed in the mixture of animal cells and the liquid of the tank 2. As shown in FIG. 7, the second gas distributor 10 may be in a shape of a roughly hollow cylinder, and a cross-section of the cylinder may be circular, elliptical, triangular, quadrilateral, or any other shape. The second gas distributor 10 may include a distributor body 11 and a second diaphragm 19 supported by the distributor body 11. The distributor body 11 may transmit the gas received from the second gas inlet unit 20 to one side of the entire second diaphragm 19. The second diaphragm 19 may dissolve and transmit the gas in a bubble-free manner into the mixture of animal cells and the liquid located on the other side of the second diaphragm 19.

As shown in FIG. 7, the distributor body 11 may include a wall 12 and a second ventilation slot 13 extending on the wall 12. The wall 12 may include a top flange 14, a bottom flange 15, and one or more protrusions 16 located between the top flange 14 and the bottom flange 15. The top flange 14, the bottom flange 15, and the protrusions 16 all protrude radially outward from an outer surface of the wall 12. The top flange 14, the bottom flange 15, and/or the protrusions 16 may form a support frame supporting the second diaphragm 19, thereby keeping the second diaphragm 19 flat.

The second ventilation slot 13 is formed in a space between the outer surface of the wall 12 and a side surface of adjacent protrusions 16, or in a space between the outer surface of the wall 12, the side surface of the protrusions 16, and a side surface of the top flange 14, or in a space between the outer surface of the wall 12, the side surface of the protrusions 16, and a side surface of the bottom flange 15. The second ventilation slot 13 is used to guide flow of the gas on the entire outer surface of the wall 12 to increase residence time of the gas in the second gas distributor 10 and promote the gas mass transfer. The second ventilation slot 13 is in fluid communication with the second gas inlet unit 20 through a gas inlet 17 of the distributor body 11 and in fluid communication with the second gas outlet unit 30 through a gas outlet 18 of the distributor body 11, thereby forming a gas flow passage through the gas inlet 17, the second ventilation slot 13, and the gas outlet 18 on the distributor body 11. The gas may flow into the second ventilation slot 13 from the gas inlet 17 and is dissolved into the mixture of animal cells and the liquid of the tank 2 through the second diaphragm 19 above the second ventilation slot 13, and the undissolved gas flows out of the second ventilation slot 13 through the gas outlet 18.

As shown in FIG. 7, the gas inlet 17 and the gas outlet 18 are tubular and may be arranged on the top flange 14. The second ventilation slot 13 may present a serpentine shape winding mainly along a horizontal direction, winding from the gas inlet 17 of the top flange 14 on a part of the outer surface of the wall 12 gradually downward to the bottom flange 15, and winding from the bottom flange 15 gradually upward to the gas outlet 18 of the top flange 14 on another part of the outer surface of the wall 12. The gas inlet 17 and the gas outlet 18 may also be arranged at other positions of the wall 12, and the second ventilation slot 13 may also be arranged on the wall 12 in various other patterns. For example, in one embodiment, the gas inlet 17 and the gas outlet 18 may be arranged on the top flange 14, the second ventilation slot 13 presents a serpentine shape winding mainly along a vertical direction on the entire outer surface of the wall 12 and is in fluid communication with the gas inlet 17 and the gas outlet 18. In another embodiment, one of the gas inlet 17 and the gas outlet 18 is arranged on the top flange 14, and the other is arranged on the bottom flange 15. The second ventilation slot 13 may be present a spiral shape extending from bottom to top around the entire outer surface of the wall 12 and in fluid communication with the gas inlet 17 and the gas outlet 18.

The second diaphragm 19 is used to dissolve and transfer gas in a bubble-free manner into the mixture of animal cells and the liquid. The second diaphragm 19 is arranged on the outer surface of the wall 12 and covers the second ventilation slot 13. The second diaphragm 19 may be fixed to the wall 12 by bonding, welding, or other means. For example, the second diaphragm 19 may be fixed to the radial outer surfaces of the top flange 14, the bottom flange 15, and/or of the protrusions 16 of the wall 12. Similar to the first diaphragm, the second diaphragm 19 may be made of silica gel, polydimethylsiloxane (PDMS), polycarbonate track-etch (PCTE), polyethylene terephthalate (PETE), polytetrafluoroethylene (PTFE), polypropylene (PP), polycarbonate (PC), nylon, polyethersulfone (PES), sintered porous material, or the like. The second diaphragm 19 may be processed with hydrophilic and positive charge or negative charge treatment so that it is not easy to be blocked due to being adsorbed by animal cells. As shown in FIG. 3A, the second diaphragm 19 may be in a form of a microporous membrane. The microporous membrane may adopt a variety of pore sizes according to different biological processes (such as stem cells, tumor cells, CHO cells, or microcarrier processes). The pore sizes are less than 0.05 μm, for example, including but not limited to 0.01 μm, 0.05 um, 0.10 μm, 0.20 μm, 0.04 μm, etc. With the above-mentioned pore size, the second diaphragm 19 may not produce bubbles when ventilated under a certain pressure. In addition, as shown in FIG. 3B, the second diaphragm 19 may also be in a form of a dense membrane. The dense membrane may be made of, for example, a silica gel material of PDMS. The dense membrane has no concept of pore sizes, but it is required to be as thin as possible in thickness, such as 50 μm to 500 μm.

Back to FIG. 6, the second gas inlet unit 20 is connected to the gas inlet 17 through a gas inlet pipeline 21 and is arranged outside the tank 2. The second gas inlet unit 20 may transmit the gas from the outside to the second ventilation slot 13 in a controlled manner. The second gas inlet unit 20 may include a second gas flow component regulator 22 (such as a mass flowmeter or a gas-proportion regulating valve) arranged on the gas inlet pipeline 21. The second gas flow component regulator 22 may regulate the flow and component ratio of inlet gas. In this embodiment, the inlet gas may be mainly composed of air, oxygen and carbon dioxide, or mainly composed of nitrogen, oxygen and carbon dioxide. A sum of the overall flow of the inlet gas is a fixed value, that is, a sum of the flow of air, oxygen, and carbon dioxide or a sum of the flow of nitrogen, oxygen, and carbon dioxide is a fixed value. The second gas flow component regulator 22 is controlled by a second controller to maintain the total flow of inlet gas at the fixed value and adjust the proportion of oxygen in the total flow in real-time, so as to adjust the dissolved oxygen concentration in the mixture.

The second gas outlet unit 30 is connected to the gas outlet 18 through a gas outlet pipeline 31 and is arranged outside the tank 2. The second gas outlet unit 30 may discharge the undissolved gas in the second ventilation slot 13 in a controlled manner to prevent the pressure increase in the second gas distributor 10 from affecting the gas control of the second gas inlet unit 20. The second gas outlet unit 30 may include a second dissolved oxygen electrode (not shown) provided in the gas outlet pipeline 31.

The second dissolved oxygen electrode may be used to detect a dissolved oxygen concentration value in the gas outlet pipeline 31 and feed it back to the second controller. The second gas outlet unit 30 may also include a pressure control valve and a sensor arranged in the gas outlet pipeline 31 to control the gas pressure (generally maintained between 0.01 mpa-0.1 mpa) in the second ventilation slot 13 of the second gas distributor 10, so as to maintain mass transfer efficiency of the second diaphragm 19.

The second solution monitoring unit 40 may include a third dissolved oxygen electrode 41 and a second pH electrode 42 electrically connected to the second controller. The third dissolved oxygen electrode 41 and the second pH electrode 42 may be arranged in a mixture of cells and the liquid in the tank 2. The third dissolved oxygen electrode 41 is used to detect the dissolved oxygen concentration value of the mixture and feed it back to the second controller. The second pH electrode 42 is used to detect the pH value of the mixture and feed it back to the second controller.

The second controller may monitor the oxygen uptake rate (OUR) consumed by animal cells in real-time through the dissolved oxygen concentration value detected by the second dissolved oxygen electrode of the second gas outlet unit 30 and the dissolved oxygen concentration value detected by the third dissolved oxygen electrode 41 of the second solution monitoring unit 40, and adjusts the oxygen proportion in the inlet gas of the second gas flow component regulator 22 of the second gas inlet unit 20. For example, the dissolved oxygen of the mixture is set to no less than 40% during a culture process, with the increase of the number of animal cells in the mixture, the oxygen flow also needs to be increased, and the proportion in the total gas flow also increases in order to maintain the same dissolved oxygen. In addition, the second controller may adjust the proportion of carbon dioxide in the inlet gas of the first gas flow component regulator 22 of the second gas inlet unit 20 through the pH value detected by the second pH electrode 42 of the second solution monitoring unit 40, so as to adjust the pH value of the mixture. For example, the pH value of the mixture is set to a fixed value during the culture process, with the continuous increase of pH value during the culture process, in order to maintain a constant pH value, the flow rate of carbon dioxide also needs to be increased, and the proportion in the total gas flow also increases.

According to the second ventilation device 1B of the present disclosure, OUR can be detected more accurately. The second ventilation device 1B adopts the design of the microporous membrane or the dense membrane, and the gas is discharged through the second ventilation device 1B, there is no tail gas buffer zone in the upper non-liquid area of the bioreactor 200. Therefore, the error in the part of OUR calculation can be reduced. The second dissolved oxygen electrode of the second gas outlet unit 30 may detect the dissolved oxygen concentration value Cout in the gas outlet pipeline 31 of the second gas outlet unit 30, and the third dissolved oxygen electrode 41 of the second solution monitoring unit 40 may detect the dissolved oxygen concentration value Csensor of the mixture in the tank 2. Therefore, the oxygen uptake rate OUR in tank 2 of the bioreactor is obtained by the following formula:

OUR=(Cout−Csensor)*KLa,

where KLA is the mass transfer coefficient of the second gas distributor 10.

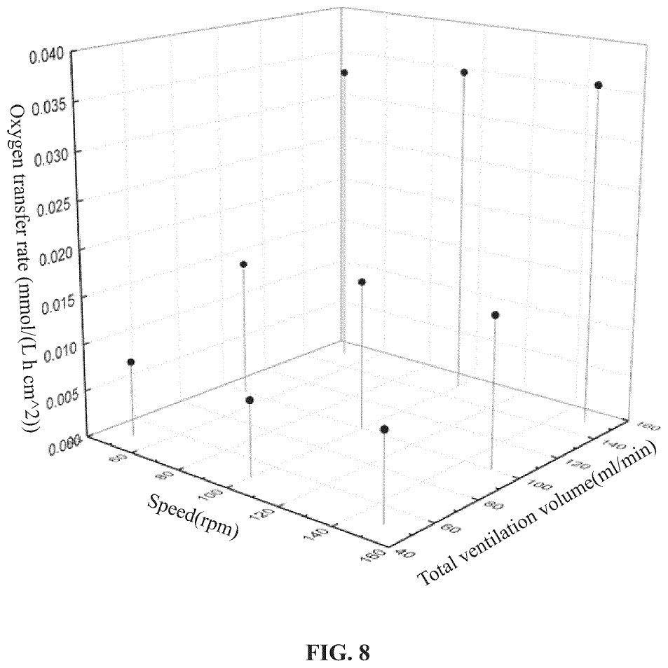

The mass transfer coefficient KLA is related to a speed of a motor driving the circulating flow of the mixture in tank 2, the total ventilation volume of the second gas distributor 10, the bubble sizes in the tank 2, and other parameters. The second ventilation device 1B according to the present disclosure adopts a bubble-free design, so no bubbles may be generated in the tank 2. FIG. 8 is a schematic diagram (taking 100 um PDMS silica gel film under atmospheric pressure as an example) illustrating an exemplary relationship between the oxygen transfer rate (a parameter proportional to mass transfer coefficient KLA), motor speed, and total ventilation volume. Since the second ventilation device 1B adopts a ventilation mode of equal ventilation volume, the total ventilation volume of the second gas distributor 10 remains unchanged no matter how the process changes. Therefore, the mass transfer coefficient KLA of the second gas distributor 10 is only related to the speed of the motor. The second controller of the second ventilation device 1B may build the relationship between the mass transfer coefficient KLA and the speed of the motor in a preset program, and the speed of the motor may not change significantly in a short time. Therefore, the second ventilation device 1B of the present disclosure may measure the mass transfer coefficient KLA of the second gas distributor 10 more accurately in advance.

Therefore, the second controller may calculate the parameter OUR through the dissolved oxygen concentration values detected by the second dissolved oxygen electrode of the second gas outlet unit 30 and the third dissolved oxygen electrode 41 of the second solution monitoring unit 40, and display a calculation result to an operator in real-time. The parameter OUR is closely related to the growth state and biomass of animal cells. These parameters may be combined with the results of cell count or living cell electrode to form key parameters such as the oxygen consumption rate of a single animal cell, which is very important for bioreactors.

The mass transfer coefficient KLA of the second ventilation device 1B according to the present disclosure is less affected by other factors, so it can be measured more accurately in advance. In addition, the total gas input of the second ventilation device 1B according to the present disclosure remains basically unchanged. Therefore, OUR may be obtained directly through a change of oxygen intake, which greatly improves the calculation accuracy of OUR.

According to the second ventilation device 1B of the present disclosure, the dissolved oxygen electrodes may be used instead of an expensive tail gas mass spectrometer detector to calculate the oxygen consumption rate, which greatly improves the monitoring level of the bioreactor for the oxygen consumption rate in the biological process and reduces the procurement cost of the expensive mass spectrometer detector.

The second ventilation device 1B according to the present disclosure uses a microporous membrane or a dense membrane to dissolve carbon dioxide, which is more conducive to the transmission effect of carbon dioxide. According to the second ventilation device 1B of the present disclosure, there will be no phenomenon that in the bubble ventilation mode, the carbon dioxide cannot be discharged in an area with high viscosity, resulting in high local carbon dioxide concentration and carbon dioxide poisoning in animal cells.

The disposable bioreactor including the second ventilation device 1A or 1B using a new microporous membrane material does not need damp-heat sterilization and only needs radiation sterilization, thereby improving the effect of oxygen transfer and achieving bubble-free mass transfer.

In some embodiments, the bioreactors 100 and 200 in the above embodiments may be integrated as a bioreactor (not shown), which includes a tank (the tank 2 in FIGS. 1 and 5) configured to contain a mixture of animal cells and a liquid, a first ventilation device (first ventilation device 1A) arranged outside the tank, and a second ventilation device (the second ventilation device 1B) arranged in the tank. In some embodiments, the bioreactor includes a dialysis component (dialysis component 10) arranged outside the tank. The dialysis component may be in fluid communication with the first ventilation device 1A. More description about the tank, the first ventilation device, the second ventilation device, and/or dialysis component may be found in elsewhere in the present disclosure, e.g., FIGS. 1-8 and description thereof. In some embodiments, the dialysis component in the bioreactor may be omitted, i.e., the bioreactor includes a tank (the tank 2 in FIGS. 1 and 5) configured to contain a mixture of animal cells and a liquid, a first ventilation device (first ventilation device 1001A) arranged outside the tank, and a second ventilation device (the second ventilation device 1B) arranged in the tank. The bioreactor in this embodiment can achieve the advantages of the bioreactors in the above embodiments, for example, improving the effect of gas transfer during cell culture process, circulating and renewing liquid separated from the animal cells, integrating the gas exchange function with the dialysis function.

Although the exemplary embodiments of the present disclosure have been described, those skilled in the art should understand that various changes and amendments can be made to the exemplary embodiments of the present disclosure without departing from the spirit and scope of the present disclosure in essence. Therefore, all changes and amendments contained in the present disclosure are within the scope of protection. The present disclosure is defined by additional claims, and equivalents of these claims are also included.

What is claimed is:

1. A bioreactor, comprising:
a tank configured to contain a mixture of animal cells and a liquid; and
a first ventilation device arranged outside the tank and including a first gas distributor, the first gas distributor being configured to transmit a gas to the liquid separated from the animal cells.

2. The bioreactor of claim 1, wherein
the liquid includes a culture medium; and
the first gas distributor includes a shell and a first diaphragm arranged in the shell, the shell of the first gas distributor includes a hollow inner cavity, and the first diaphragm separates the hollow inner cavity of the shell into a culture medium chamber through which the culture medium flows and a gas chamber through which the gas flows, the culture medium chamber of the first gas distributor is in fluid communication with the tank through a liquid outlet pipe, and the first diaphragm dissolves the gas in the gas chamber into the culture medium in the culture medium chamber without bubbles.

3. The bioreactor of claim 2, wherein
the first diaphragm is arranged to make the culture medium chamber and the gas chamber in a form of an inner cylinder and an outer cylinder nested with each other.

4. The bioreactor of claim 2, wherein
the gas chamber includes a first ventilation slot arranged along an inner surface of a sidewall of the shell, the first ventilation slot being configured to guide an inlet gas to flow on the entire inner surface of the sidewall;
the first diaphragm is arranged on the inner surface of the sidewall and covers the first ventilation slot.

5. The bioreactor of claim 4, wherein
the sidewall comprises a plurality of convex parts, and the plurality of convex parts protrude radially inward from the inner surface of the sidewall; and
the first ventilation slot is formed in a space between the inner surface of the sidewall and side surfaces of adjacent convex parts.

6. The bioreactor of claim 2, wherein
the first diaphragm is in a form of a dense membrane or a microporous membrane,
a pore size of the microporous membrane ventilates the first diaphragm without bubbles under a certain pressure, and
a thickness of the dense membrane is between 50 μm to 500 μm.

7. The bioreactor of claim 2, wherein
the first ventilation device further includes a first gas inlet unit, which is in fluid communication with the gas chamber through a gas inlet pipe and transmits the inlet gas to the gas chamber, and a first gas outlet unit, which is in fluid communication with the gas chamber through a gas outlet pipe and discharges an undissolved gas in the gas chamber from the first gas distributor.

8. The bioreactor of claim 7, wherein
the first gas inlet unit includes a first gas flow component regulator arranged on the gas inlet pipe, the first gas flow component regulator being configured to adjust a component proportion of the inlet gas; and
the first gas outlet unit includes a pressure control valve and a sensor arranged in the gas outlet pipe to control gas pressure in the gas chamber.

9. The bioreactor of claim 7, wherein the first ventilation device further includes
a first dissolved oxygen electrode arranged in the mixture, the first dissolved oxygen electrode detecting a dissolved oxygen concentration value in the mixture; and
a first controller, the first controller adjusting a proportion of oxygen in the inlet gas of the first gas flow component regulator through the dissolved oxygen concentration value detected by the first dissolved oxygen electrode, so as to adjust the dissolved oxygen concentration value of the mixture in the tank.

10. The bioreactor of claim 9, wherein
the first ventilation device further includes a first pH electrode arranged in the mixture, the first pH electrode being configured to detect a pH value in the mixture; and
the first controller is configured to adjust a proportion of carbon dioxide in the inlet gas of the first gas flow component regulator through a pH value detected by the first pH electrode, so as to adjust the pH value of the mixture.

11. The bioreactor of claim 2, further comprising a dialysis component, which is arranged outside the tank and comprises a dialysis filter, wherein the dialysis filter is configured to dialyze harmful metabolites in the culture medium separated from the animal cells into a dialysate; and the dialysis component is in fluid communication with the first ventilation device, and the first gas distributor and the dialysis filter are formed as an integral part.

12. The bioreactor of claim 11, wherein
the dialysis filter comprises a shell, a filter element arranged in the shell, and a hollow inner cavity, the shell of the dialysis filter is in a shape of a cylinder with a top wall, a bottom wall and a sidewall extending between the top wall and the bottom wall, and the filter element separates the hollow inner cavity of the shell of the dialysis filter into a culture medium chamber through which the culture medium flows and a dialysate chamber through which the dialysate flows; and
the culture medium chamber of the dialysis filter and the culture medium chamber of the first gas distributor are in fluid communication through a connecting pipe.

13. The bioreactor of claim 12, wherein the connecting pipe and at least one of a corresponding part of the shell of the dialysis filter or a corresponding part of the shell of the first gas distributor are arranged to be transparent so that a flow state of the culture medium in the connecting pipe can be observed.

14. The bioreactor of claim 12, wherein the culture medium chamber of the dialysis filter is in fluid communication with the tank through a liquid inlet pipe; the dialysis component further comprises a fresh dialysate storage tank and a waste dialysate storage tank, both of which are in fluid communication with the dialysate chamber; the dialysis filter is arranged on a pipe between the dialysate chamber and the fresh dialysate storage tank for filtering out insoluble particles in the dialysate.

15. The bioreactor of claim 11, further comprising a cell separation device configured to separate the culture medium from the animal cells, and the cell separation device is arranged in the tank and is in fluid communication with the dialysis filter.

16. The bioreactor of claim 1, further comprising a second ventilation device arranged in the tank and configured to transmit the gas to the mixture in the tank.

17. The bioreactor of claim 16, wherein the second ventilation device includes a second gas distributor, the second gas distributor is configured to be immersed in the mixture of the tank and includes a distributor body and a second diaphragm supported by the distributor body, wherein the distributor body is provided with a second ventilation slot, and the second diaphragm is configured to dissolve the gas in the second ventilation slot into the mixture without bubbles.

18. The bioreactor of claim 17, wherein the second ventilation device includes a second gas inlet unit, which is in fluid communication with the second ventilation slot and transmits the inlet gas to the second ventilation slot, and a second gas outlet unit, which is in fluid communication with the second ventilation slot and discharges an undissolved gas in the second ventilation slot from the distributor body.

19. The bioreactor of claim 18, wherein
the second gas inlet unit is in fluid communication with the second ventilation slot through a gas inlet pipeline, the second gas outlet unit is in fluid communication with the second ventilation slot through a gas outlet pipeline; and
the second gas inlet unit includes a second gas flow component regulator arranged on the gas inlet pipeline and electrically connected to a second controller, the second gas flow component regulator is configured to maintain a total flow of the inlet gas at a constant value and adjust a component proportion of the inlet gas according to a command of the second controller.

20. The bioreactor of claim 19, wherein the second controller is configured to monitor and adjust an oxygen uptake rate OUR consumed by the animal cells in the tank in real-time, the oxygen uptake rate OUR is determined by a calculation formula of
OUR=(Cout−Csensor)*KLa,
where Cout denotes a dissolved oxygen concentration value detected by a second dissolved oxygen electrode which is arranged in the gas outlet pipeline; Csensor denotes a dissolved oxygen concentration value detected by a third dissolved oxygen electrode arranged in the mixture; and KLA is a mass transfer coefficient of the second gas distributor, wherein the mass transfer coefficient of the second gas distributor is related to a rotational speed of a motor in the tank, the motor driving the circulating flow of the mixture.
